(19) United States
(12) Patent Application Publication
(10) Pub. No.: US 2010/0184029 A1
(43) Pub. Date: Jul. 22, 2010

(54) METHODS AND COMPOSITIONS FOR IDENTIFYING BIOMARKERS USEFUL IN CHARACTERIZING BIOLOGICAL STATES

(76) Inventors: James C. Willey, Toledo, OH (US); Erin L. Crawford, Rossford, OH (US); Thomas M. Blomquist, Perrysburg, OH (US)

Correspondence Address: WILSON, SONSINI, GOODRICH & ROSATI, 650 PAGE MILL ROAD, PALO ALTO, CA, 94304-1050, US

(21) Appl. No.: 12/410,399

(22) Filed: Mar. 24, 2009

Related U.S. Application Data
(63) Continuation-in-part of application No. 11/470,192, filed on Sep. 5, 2006.
(60) Provisional application No. 60/713,628, filed on Sep. 2, 2005.
(60) Provisional application No. 60/713,629, filed on Sep. 2, 2005.
(60) Provisional application No. 60/714,138, filed on Sep. 2, 2005.
(60) Provisional application No. 61/039,410, filed on Mar. 25, 2008.

(51) Int. Cl. C12Q 1/68

(52) U.S. Cl. 435/6

(57) ABSTRACT

The present invention relates to methods, compositions, and kits for identifying biomarkers useful in characterizing biological states. In particular, the invention relates to methods and compositions for molecular characterization of biological states by gene expression profiling. The invention also relates to assessing effects of DNA polymorphisms on regulation of transcription. The biomarkers and polymorphisms identified find use in diagnostic and treatment approaches, e.g., some embodiments of the invention provide methods and kits for detecting bronchogenic carcinoma and risks thereof.

### CROSS-REFERENCE

This application is a continuation-in-part application of Ser. No. 11/470,192 filed on Sep. 5, 2006, which claims the benefit of U.S. Provisional Application No. 60/713,628, 60/713,629 and 60/714,138, all of which were filed on Sep. 2, 2005 and all of which are incorporated herein by reference in their entirety.

This application claims the benefit of U.S. Provisional Application No. 61/039,410, filed Mar. 25, 2008, which is incorporated herein by reference in its entirety.

### STATEMENT AS TO FEDERALLY SPONSORED RESEARCH

This invention was made with the support of the United States government under Contract number R24 CA 95806.

### BACKGROUND OF THE INVENTION

Molecular characterization of a particular biological state to improve prognosis, therapeutic selection, and clinical outcomes has been a dominant paradigm for many years. A common limitation of such studies is the lack of readily deployable test kits with the accuracy, low RNA requirement, and inter-site concordance required for routine clinical use. Another dominant paradigm is assessing the correlation between a particular variation in DNA sequence, or polymorphism, and risk for a particular condition. This has been a dominant paradigm for many years. A common limitation of such studies, however, is that they involve assessment of a single polymorphism or occasionally, a few polymorphisms. Further, although the polymorphism assessed typically resides within a gene associated with a particular biological state, the selection of a polymorphism for study can be largely empiric, e.g., not being based on known function. When a gene with low prior likelihood is assessed for association with a biological state, a statistically valid assessment may require very large study populations, so large as to be impractical. More recently, Genome Wide Association Studies (GWAS) involving assessment of hundreds of thousands of polymorphisms for association with a biological state are being conducted with increasing frequency. In part, this approach is based on the consensus opinion that multiple polymorphisms at widely divergent sites in the genome may contribute to a biological state, and if a large enough number of SNPs is assessed using large enough sample sizes, significant associations can be discovered. There are limitations to this approach. For example, for any particular gene, multiple infrequent polymorphisms at different sites, may contribute to disease risk, rather than a single frequent polymorphism. It is difficult to appropriately assess the risk contribution of multiple infrequent polymorphisms unless sample sizes are in the many thousands. In addition, these studies are based on assumptions regarding linkage of contiguous regions (haplotype blocks) and these assumptions can be incorrect. Further, the polymorphisms included in GWAS are typically focused on coding regions, but there is increasing evidence that regulatory region polymorphisms may play a larger role in determining biological states. Thus, there remains a need for a new approach to identify biomarkers that can diagnose undesirable conditions and serve as therapeutic targets.

Bronchogenic carcinoma (BC) is an example of a condition wherein risk of acquisition may be influenced by multiple polymorphisms. BC is the leading cause of cancer-related death in the United States. While cigarette smoking is the primary risk factor, only some heavy smokers acquire the disease. Cigarette smoking is also the primary cause of other pulmonary conditions such as chronic obstructive pulmonary disease (COPD). COPD is one of the most common chronic conditions and the fourth leading cause of death in the United States. Identifying those at greater risk for BC and/or COPD can enhance development of methods and compositions for early detection, as well as methods and compositions for treating and/or preventing the disease. The instant invention relates to such methods and compositions for identifying individuals at risk for BC and/or COPD, as well as other biological states, including e.g., other cancer and/or other lung-related conditions.

### SUMMARY OF THE INVENTION

In one aspect of the invention a method is disclosed for characterizing gene expression comprising:(a) amplifying one or more native templates in a solution comprising a standardized mixture of internal standards and said one or more native templates to produce one or more first amplicon(s); (b) amplifying said one or more first amplicons in a nanofluidic device, said nanofluidic device comprising at least one labeled probe that binds with greater affinity to either a first amplicon amplified from said native template or a first amplicon amplified from said internal standard; (c) detecting signal from said at least one labeled probe bound to said native template and to said internal standard, and measuring a ratio of said native template to said internal standard based on said detected signals; and (d) determining an amount of said one or more first amplicons from said ratio by multiplying said ratio by the number of copies of said standardized mixture of internal standards. In one embodiment the native template is DNA. In another embodiment the native template is mRNA. In another embodiment the native template is cDNA. In another embodiment the amplification to produce said one or more first amplicon(s) is a competitive amplification of said native template and said standardized mixture of internal standards. In another embodiment at least two labeled probes are used, wherein a first labeled probe binds with greater affinity to said first amplicon amplified from said native template and wherein a second labeled probe binds with greater affinity to said first amplicon amplified from internal standard. In another embodiment the at least two labeled probes are fluorescently labeled. In another embodiment at least two labeled probes comprise different labels. In another embodiment a signal generated from the probe bound to said first amplicon amplified from said native template or said first amplicon amplified from said internal standard is detected and quantified.

In another aspect of the invention a method is disclosed for A method to assess an effect of DNA polymorphisms on regulation of transcription comprising: (a) amplifying a native template from an individual using a first allele specific primer that anneals to a first allele at a first polymorphic DNA region and a non-allele specific primer to produce a first allele-specific amplification product; (b) amplifying a native template from said individual using a second allele specific primer that anneals to a second allele at a first polymorphic DNA region and said non-allele specific primer to produce a second allele-specific amplification product; (c) determining a ratio of each allele-specific product to the other; (d) amplifying a DNA template of said individual using said first allele-specific primer for the first polymorphic DNA region and a second non-allele specific primer to produce a third amplicon spanning a sequence that contains a first allele at a second polymorphic region that is collinear with the first allele at the first polymorphic region; (e) amplifying a DNA template of said individual using said second allele-specific primer that binds to the second allele at the first polymorphic site and said second non-allele specific primer to produce a fourth amplicon spanning a sequence that contains a second allele at said second polymorphic region that is collinear with said second allele at said first polymorphic region; (f) determining a sequence of said third or fourth amplicon; and (g) using said sequence to assess an effect of said second DNA polymorphic region on regulation of allele-specific transcription measured through allele-specific cDNA priming of said first polymorphic region. In one embodiment the first and second allele specific amplicons span a polymorphic locus putatively responsible for regulating transcription, translation, splicing, and/or degradation. In another embodiment the sequence collinear with said first polymorphic region is in the 5′ non-transcribed, intronic, or 3′ non-transcribed region of a DNA template. In another embodiment the DNA template is amplified with said first allele-specific primer for said first polymorphic site and a second primer that spans a second polymorphic locus in the 5′ non-transcribed region, 3′ non-transcribed region, or an intron. In another embodiment the one or more internal standards corresponding to each allele is generated to ensure specificity of the allele-specific primers comprising: synthesizing a primer for production of a first shortened allele-specific internal standard corresponding to the native template in (a); synthesizing a primer for production of a second shortened allele-specific internal standard corresponding to the native template in (b); producing a serial dilution of said first internal standard and combining it with a constant amount of the native template in (a) to ensure allele-specificity of the primer; and producing a serial dilution of said second internal standard and combining it with a constant amount of the native template in (b) to ensure allele-specificity of the primer. In another embodiment the first and second internal standards are mixed together at known concentrations relative to each other. In another embodiment the first and second internal standards are mixed with a known concentration of an internal standard for one or more genes. In another embodiment the internal standard for one or more genes is a loading control. In another embodiment the method further comprises measuring the transcript abundance of each allele relative to a known quantity of a corresponding internal standard. In another embodiment the native template is DNA. In another embodiment the native template is mRNA. In another embodiment the native template is cDNA.

In another aspect of the invention a kit is disclosed for assessing the effect of DNA polymorphisms on regulation of transcription comprising: a) a first allele-specific primer that anneals to a first allele at a first polymorphic region in one or more native templates and a second primer suitable for PCR amplification; b) a third allele-specific primer that anneals to a second allele at said first polymorphic region; c) a fourth primer that binds to a nucleic acid sequence and in combination with said first allele-specific primer or said third allele-specific primer produce an amplicon that spans a DNA sequence containing a second polymorphic region that is a collinear transcribed sequence, 5′ non-transcribed sequence, an intronic sequence, or a 3′ non-transcribed sequence of a cDNA sample or genomic DNA sample; and d) instructions for use. In one embodiment the instructions disclose the use of allele-specific primers for amplifying each native template in a sample from an individual, and the third primer in the amplification of a genomic DNA native template of said sample.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE FIGURES

The novel features of the invention are set forth with particularity in the appended claims. A better understanding of the objects, features and advantages of the present invention will be obtained by reference to the following detailed description that sets forth illustrative embodiments, in which the principles of the invention are utilized, and the accompanying drawings of which:

FIG. 1 illustrates the Standardized Nanoliter Array PCR (SNAP) Gene Expression Signature Test.

FIG. 2 illustrates the reproducibility and robustness of the preamplification StaRT-PCR™ method.

FIG. 3 illustrates the utility of a two step StaRT-PCR in allowing the use of a small sample for SNAP assays.

FIG. 4(a-b) illustrates the feasibility of using formalin fixed paraffin embedded (FFPE) RNA in SNAP assays.

FIG. 5 illustrates the Linked Allele Specific Transcript Abundance and Sequencing (LASTAS) assay.

FIG. 6 illustrates the preparation of C and T allele-specific primers for the LASTAS assay.

FIG. 7 illustrates the sequencing results from a LASTAS assay.

FIG. 8 illustrates applications of LASTAS.

FIG. 9 illustrates measuring allele-specific Gene Expression

FIG. 10 illustrates allele-specific standardized reverse transcription PCR methodology.

FIG. 11(A-B) illustrate testing for allelic specificity.

FIG. 12 illustrates Inter-individual variation in allelic imbalance of XPG.

FIG. 13 illustrates inter-sample comparison of allelic data.

FIG. 14 illustrates results obtained from seven lung epithelial cell cDNA samples.

FIG. 15 illustrates CEBPG transcription factor correlation with XPG in normal and cancerous tissue.

FIG. 16 illustrates allele-specific sequencing.

FIG. 17 illustrates results obtained from seven lung epithelial cell cDNA samples.

FIG. 18 illustrates results obtained from a Chromatin Immuno-precipitation assay.

FIG. 19 illustrates a flow-chart of the 2-phase model.

FIG. 20 illustrates a comparison of ERCC5 TA variance to genotype.

### DETAILED DESCRIPTION OF THE INVENTION

The present invention relates to methods and compositions for molecular characterization of a particular biological state, in particular molecular characterization by gene expression profiling (hereinafter “GEP”) on a nanoplatform system. Additionally the present invention relates to assessing the effects of DNA polymorphisms on regulation of transcription by relating the amount of allele-specific transcription product to the collinear allele at a polymorphic site through linked allele-specific transcript abundance and sequencing analysis. The GEP protocol and polymorphisms identified find use in diagnosis prognosis, therapeutic selection, and clinical outcomes, e.g., in some embodiments the invention provides methods and kits for detecting BC and risks thereof.

I. Methods and Compositions for Gene Expression Profiling using Standardized NanoArray PCR (SNAP)

In one aspect, the invention relates to methods for characterizing gene expression on a nanoplatform system. In some embodiments, the method involves amplifying nucleic acids in a solution comprising native templates and respective internal standards within a standardized mixture of internal standards (SMIS) to form one or more first amplicon(s). In a preferred embodiment, the said amplification is a competitive amplification of one or more native templates and respective internal standards within the SMIS. In some embodiments, the method further involves secondarily amplifying in a nanofluidic device one or more amplicon(s) from a first amplification. In some embodiments the nanofluidic device is the OpenArray system by BioTrove, Inc. In some embodiments gene expression profiling is carried out to perform molecular characterization of a biological state.

“Native template” as used herein can refer to nucleic acids extracted from a case sample. In some embodiments the native template is genomic DNA. In other embodiments the native template is mRNA. In yet other embodiments the native template is cDNA.

A “biological state” as used herein can refer to any phenotypic state, for e.g., a clinically relevant phenotype or other metabolic condition of interest. Biological states can include, e.g., a disease phenotype, a predisposition to a disease state or a non-disease state; a therapeutic drug response or predisposition to such a response, an adverse drug response (e.g. drug toxicity) or a predisposition to such a response, a resistance to a drug, or a predisposition to showing such a resistance, etc. In some embodiments, the drug may be an anti-tumor drug.

“Standard mixture of internal standards” (SMIS) as used herein can refer to a mixture comprising a number of internal standards, e.g., a number of competitive templates. A known quantity of internal standard for each of multiple genes can be combined in a SMIS. Measuring each gene relative to a known number of internal standard molecules within a SMIS in each reaction controls for variation in amount of sample loaded into the reaction, and controls for unpredictable inter-sample variation in the efficiency of the PCR caused by reagent consumption, PCR inhibitors, and/or product inhibition. SMIS controls for preferential amplification of one transcript over another due to differences in amplification efficiencies. Preparation and use of standardized mixtures are described in U.S. patent application Ser. Nos. 11/072,700 and 11/103,397, which are herein incorporated by reference in their entirety.

“Amplicon(s)” as used herein means a molecule of nucleic acid that has been synthesized using amplification techniques

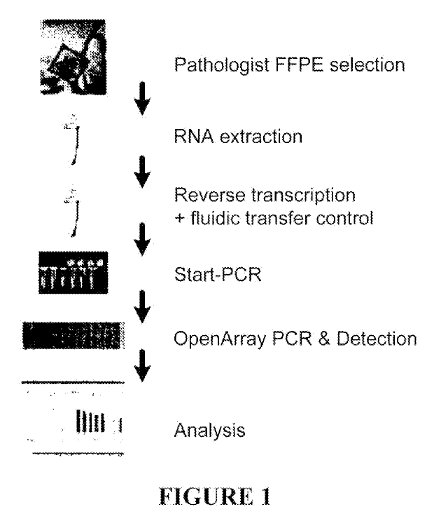

FIG. 1 illustrates one embodiment for performing the SNAP assay.

First, sample pathology is confirmed and tumor enriched sections are selected for native template extraction. In a preferred embodiment the native template is mRNA.

In some embodiments, a plurality of case samples and reference samples are used. A plurality refers to, e.g., 2 or more. Preferably more than about 10 case samples are used. Preferably more than 11 case samples, more than 12 case samples, more than 13 case samples, more than 14 case samples, and more than 15 case samples are used. In a preferred embodiment, 16 case samples are used. Preferably more than 2 reference samples and more than 3 reference samples are used. In a preferred embodiment, 4 reference samples are used.

Case samples can be selected from a variety of sources including but not limited to, a formalin fixed paraffin embedded (FFPE) block slice; a fine needle aspirate (FNA) of suspected a cancer lesion; a swab of culture; a brush of epithelial cells; a pinch of tissue; a biopsy extraction; a biological fluid; anatomically small, but functionally important tissues of the brain, developing embryo tissues, animal tissues, and laser captured micro-dissected samples. In some embodiments a tissue can be selected from an organ, skin, a tumor, a lymph node, an artery, an aggregate of cells and/or an individual cell. Biological fluids can include, e.g., saliva, tears, mucus, lymph fluids, sputum, stool, pleural fluid, pericardial fluid, lung aspirates, exudates, peritoneal fluid, plasma, blood, serum, white blood cells, cerebral spinal fluid, synovial fluid, amniotic fluid, milk, semen, urine, and the like, as well as cell suspensions, cell cultures, or cell culture supernatants. Samples may be crude samples or processed samples, e.g., obtained after various processing or preparation steps. In some embodiments various cell separation methods (e.g., magnetically activated cell sorting) can be applied to separate or enrich analytes of interest in a biological fluid, such as blood. In some embodiments the cell separation methods can include one or more of the following, fluorescence activated cell sorting, flow cytometry, centrifugation, gradient centrifugation, filtration, size based selection, serial dilution, selective cell lysis (e.g. lysis of enucleated red blood cells). A sample may also comprise a dilution, e.g., diluted serum or dilutions of other complex and/or protein-rich mixtures. Preferred embodiments of the present invention can be practiced using small starting materials to yield quantifiable results.

At steps 2 and 3, in one embodiment, mRNA from the case sample is extracted and reverse transcribed to synthesize cDNA according to well established protocols.

At step 4, nucleic acids from the case samples are distributed between PCR tubes containing primers for gene targets and serial dilution of SMIS. In one embodiment StaRT-PCR™ tubes (e.g., six tubes) are used. In a preferred embodiment the SMIS serial dilution is 10-fold. In other embodiments, the dilution can be 2-fold, 3-fold, 4-fold, 5-fold, 6-fold 7-fold, 8-fold, 10-fold, or more than 10-fold. Samples are subjected to PCR to form one or more amplicon(s). In a preferred embodiment, the samples are subjected to thirty five cycles of PCR. In other embodiments, the samples are subjected to at least twenty cycles, at least twenty five cycles, or at least thirty cycles of PCR. The nucleic acids analyzed from the case samples can refer to an mRNA transcript or a cDNA obtained from the mRNA.

At step 5, the amplicon(s) from step 4 are diluted and transferred in to a system that allows for parallel low-volume solution phase reactions to be performed. In one embodiment the system is automated. In a preferred embodiment system is the OpenArray system made by BioTrove, Inc. which is preloaded with amplification primers and at least two differentially labeled probes specific for either native template or internal standard. In a preferred embodiment, said probes are fluorescently labeled. In a preferred embodiment, the amplicon(s) from step 4 are diluted 100-fold. In other embodiments, the amplicon(s) from step 4 are diluted 10-fold, 20-fold, 30-fold, 40-fold, 50-fold, 60-fold, 70-fold, 80-fold, or 90-fold. The diluted amplicon(s) from step 4 are further amplified by using a quantitative PCR system. In one embodiment the PCR system is the TaqMAN PCR system, which is used in conjunction with the OpenArray system. In a preferred embodiment, the amplicon(s) are subjected to thirty five cycles of PCR. In other embodiments, the amplicon(s) are subjected to at least twenty cycles, at least twenty five cycles, or at least thirty cycles of PCR.

The OpenArray system is a nanofluidic PCR device manufactured by BioTrove, Inc. It is a high density array of nanoliter-scale through-holes or chambers for implementing up to 3072 PCR analyses with 33 nl per reaction in an array the size of a microscope slide.

At step 6, following the TaqMAN PCR from step 5, the ratios of the signal from labeled probes are measured, and native template concentration estimated from said signal ratio versus [internal standard] curve, according to well established protocols. Reference gene copies are used to correct for loading of sample into the StaRT-PCR reaction prior to GEP calculation.

In one embodiment a two-step StaRT-PCR™ method is performed, wherein the second PCR step is performed in a nanofluidic OpenArray system, which is used to perform highly scalable GEP. This allows readily deployable accurate test kits with the low RNA requirement to be used. In one embodiment the kits provide accurate results with sufficient level of reproducibility to meet standards of inter site concordance required for routine clinical use. Through the first round of amplification with multiple sets of primers and internal standards in the same reaction, multiple gene expression measurements are obtained from RNA quantities that normally yield only one GEP measurement. The data presented here support the utility of this paradigm in molecular characterization of a biological state by GEP, as provided below.

Selection of Genes for Incorporation into the OpenArray Assays

In another embodiment, genes that may have prognostic value in early stage lung cancer patients are chosen. However, any genes of interest can be chosen for incorporation into the OpenArray assays. The prognostic value of tumor gene expression profiles in cancer patients is well established. Many such genes have been identified in lung cancer. See Chen M, et al., N. England J. Med., 356(1), 11-20, 2007; PottiA, et al., N. England J. Med., 355(6), 570-80, 2006; Beer D G, et al., Nat. Med. 8(8), 816-24, 2002; Bhattacharjee A, et al., Proc. Natl. Acad. Sci, USA, 98(24), 13790-5, 2001; and Garber M E, et al., Proc. Natl. Acad. Sci, USA, 98(24), 13784-9, 2001, which are herein incorporated by reference in their entirety. Of these, the five gene signature identified by Chen M et al. (2007), has been found to be prognostic in lung cancer patients. These five genes were derived form a larger set of sixteen. See Chen M, et al., (2007). In one preferred embodiment, these sixteen genes are chosen to be incorporated into the OpenArray assay and are listed in Table 1.

TABLE 1

| Gene | UniGene ID |
|---|---|
| ERBB3 | HS.118681 |
| LCK | Hs.470627 |
| DUSP6 | Hs.298654 |
| STAT1 | Hs.470943 |
| MMD | Hs.463483 |
| CPEB4 | Hs.127126 |
| RNF4 | Hs.66394 |
| STAT2 | Hs.530595 |
| NF1 | Hs.113577 |
| FRAP1 | Hs.338207 |
| DLG2 | Hs.503453 |
| IRF4 | Hs.401013 |
| ANXA5 | Hs.480653 |
| HMMR | Hs.72550 |
| HGF | Hs.396530 |
| ZNF264 | Hs.515634 |

### Design of Primers for SNAP

In a preferred embodiment, the primer design criteria for FFPE sample amplification as described in Cronin M, et al., AJP 164(1), 35-42, 2004 are used.

One of skill in the art will recognize that the methods provided herein can be applied to the molecular characterization by GEP of other cancer-related conditions. Examples of other cancer-related conditions include, but are not limited to, breast cancer, skin cancer, bone cancer, prostate cancer, liver cancer, lung cancer, brain cancer, cancer of the larynx, gallbladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neurons, intestinal ganglioneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythermia vera, adenocarcinoma, glioblastoma multiforma, leukemias, lymphomas, malignant melanomas, epidermoid carcinomas, and other carcinomas and sarcomas.

In some embodiments, case samples may be obtained from different stages of cancer. Cells in different stages of cancer, for example, include non-cancerous cells vs. non-metastasizing cancerous cells vs. metastasizing cells from a given patient at various times over the disease course. Cancer cells of various types of cancer may be used, including, for example, a bladder cancer, a bone cancer, a brain tumor, a breast cancer, a colon cancer, an endocrine system cancer, a gastrointestinal cancer, a gynecological cancer, a head and neck cancer, a leukemia, a lung cancer, a lymphoma, a metastases, a myeloma, neoplastic tissue, a pediatric cancer, a penile cancer, a prostate cancer, a sarcoma, a skin cancer, a testicular cancer, a thyroid cancer, and a urinary tract cancer.

One of skill in the art will recognize that the methods provided herein can be applied to the identification of biomarkers for other lung-related conditions. Examples of lung-related conditions include, e.g., sarcoidosis, pulmonary fibrosis, pneumothorax, fistulae, bronchopleural fistulae, cystic fibrosis, inflammatory states, and/or other respiratory disorders. Lung-related conditions can also include smoking-related and/or age-related changes to the lung, as well as lung damage caused by a traumatic event, infectious agents (e.g., bacterial, viral, fungal, tuberculin and/or viral agents), exposure to toxins (e.g., chemotherapeutic agents, environmental pollutants, exhaust fumes, and/or insecticides), and/or genetic factors (e.g., alpha-1 antitrypsin deficiency and other types of genetic disorders which involve elastic and/or connective tissues degradation and/or impaired synthesis of elastic and/or connective tissues and/or impaired repair of elastic and/or connective tissues of the lungs).

### II Methods and Compositions for Assessing an Effect of DNA Polymorphisms on Regulation of Transcription Using Linked Allele-Specific Transcript Abundance and Sequencing (LASTAS) Method

In another aspect, the invention relates to methods for assessing an effect of DNA polymorphisms on regulation of transcription. In some embodiments, the method involves amplifying an internal standard template (IS) and a native template from a cDNA sample using an allele-specific primer paired with a second primer, and determining the amount of said allele-specific amplification product. In other embodiments, the method further involves amplifying a native template from a genomic DNA sample using said allele-specific primer and a primer in the 5′ non-transcribed, intronic, or 3′ non-transcribed region of said genomic DNA template, and sequencing said amplified DNA, thus enabling assessment of correlation of each allele at a polymorphic site with abundance of transcript from the respective collinear coding region. In some embodiments LASTAS is carried out to assess the effect of DNA polymorphisms on regulation of transcription of genes involved in a biological state.

For example, some embodiments provide a method for identifying DNA sequence variation associated with disease and/or risk for disease involving a) determining expression levels of genes involved in i) conferring the phenotype or ii) regulating transcription of the genes involved in conferring the phenotype, and b) identifying one or more DNA sequence variations responsible for determining regulation of the involved genes.

“Polymorphism” or “DNA sequence variation” as used herein can refer to any one of a number of alternative forms of a given locus (position) on a chromosome. The alternative form may involve a single base pair difference, such as a single nucleotide polymorphism (SNP). In some embodiments, the polymorphism may involve more than one base pair change, e.g., it may involve at least about 2, at least about 3, or least about 10 nucleotide differences. In some embodiments, the polymorphism may involve less than about 50, less than about 100, less than about 200, or less than about 500 nucleotide differences. The term polymorphism may also be used to indicate a particular combination of alleles, e.g., two or more SNPs, at a particular locus. In some embodiments, identification of a particular nucleotide variation at each of multiple loci identifies a biological state, e.g., a specific combination of alleles at in a single or multiple particular genes may indicate risk for a disease condition.

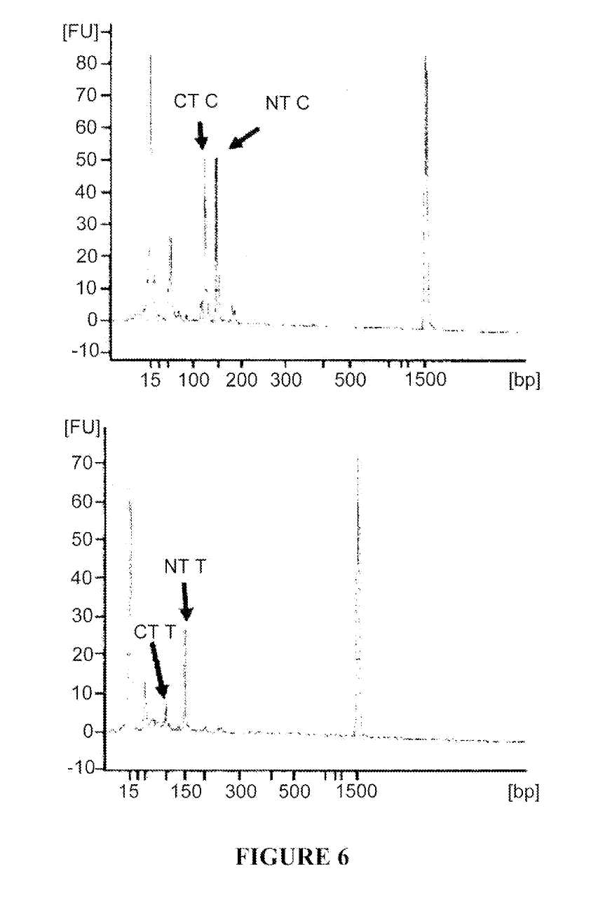

FIG. 6 is a schematic diagram of the LASTAS assay in some embodiments disclosed herein.

For example in order to conduct LASTAS analysis of the common E2F/YY1 transcription factor polymorphic binding site (A or G) at +25 relative to the TSS-2 site of ERCC5, reverse primers specific to allele C or T at polymorphic site in the 5′ region are used for sequencing of genomic DNA (with −440 primer) and for transcript abundance measurement of cDNA native template (NT) relative to known number of IS molecules (using +92 primer). With allele-specific primers, for each individual the allele at the polymorphic +25 E2F/YY1 binding site is linked to abundance of transcript derived from the collinear coding region. Panels B-E are schematics demonstrating electropherograms of non-specific or specific PCR products for the IS and corresponding NT of some embodiments. In this example, the IS for T allele is shorter than the IS for C allele. Panel B demonstrates the electropherogram when primers for both alleles are present. Panel C demonstrates theoretical products when a candidate C allele-specific primer is used but it is not specific, yielding both C allele IS and NT products, but also some of the T allele IS product. Panels D and E represent products if each allele-specific primer performs well. Once appropriate quality control is done to ensure allele-specificity, the primers may be used for both transcript abundance measurement and sequencing.

ERCC5 is a nucleotide excision repair enzyme essential for removing DNA adducts associated with cigarette smoke components.

“Region” as used herein can refer to a nucleic acid sequence that preferably involves fewer base pairs than the entire gene. A region can include coding and non-coding, transcribed and non-transcribed, and/or translated and un-translated regions. For example, a region of a gene can include the regulatory elements 5′ of the coding region, e.g., recognition sites for one or more transcription factors (TF). A region for TF binding can include 5′ non-transcribed regions, 3′ non-transcribed regions and/or coding regions. Methods provided herein teach specific region to focus on in identification of polymorphisms indicative of a biological state. In some embodiments, the region spans at least about 5, at least about 10, at least about 20, at least about 30, at least about 50, at least about 80, or at least about 100 bases. In some embodiments, the region spans less than about 150, less than about 200, less than about 250, less than about 300, or less than about 500 bases.

A shortened IS for each allelic NT is designed such that it is amplified with the same efficiency as the respective NT by the allele-specific primers. A shortened IS for each allele is created through PCR amplification, then mixed together in equimolar concentrations. The IS, is necessary to a) ensure that each primer amplifies specifically in each reaction and thereby control for false positives, b) to control for PCR inhibitors and other interfering substances and thereby control for false negatives.

In one example, the LASTAS PCR method was used to measure quantitative transcript abundance levels derived from each parental gene copy within the same tissue sample, and link transcript abundance from each parental gene copy with cis-acting sequence variations on the same chromosome. Specificity testing of allele-specific primers demonstrated less than 5% non-targeted allele PCR amplification even when non-targeted allele was in 10-fold excess at start of PCR. These allele-specific primers were used to determine accuracy of estimates based on haplotype block assumptions for linkage between a SNP in the regulatory region (177 bp away) of ERCC5 and a SNP in the 5′-UTR of ERCC5. Specifically, each allele-specific primer was used to sequence the region containing these two SNPs in 71 individuals. There were two alleles at the regulatory region SNP (G and A) and two alleles at the 5′-UTR SNP (C and T). There were eight double heterozygotes with G and C on one chromosome and A and T on the other chromosome. Notably, 10% of chromosomes with A at the regulatory region SNP had allelic reversal at the 5′-UTR SNP (A-C). Based on this measured frequency, it is predicted that in 7.4% of doubly heterozygous individuals there will be reversal of allelic linkage from that predicted by haplotype map estimates. Additionally, four novel, and potentially important sequence polymorphisms, were discovered within this region and located to a parental chromosome using allele-specific sequencing. These data demonstrate that LASTAS may be used to directly measure the association of a particular allele at a cis-polymorphic site with transcript abundance from the same chromosome. In the case of ERCC5, this is predicted to eliminate what would be a >7.4% reversal error in associating allele with transcript abundance if only haplotype mapping estimates were used.

### Design of Primers for LASTAS

Allele-specific primers are designed such that they specifically bind to one allele at a heterozygous polymorphic site in the transcribed region that is within sequencing distance of another polymorphic site of interest. In a preferred embodiment, the primers are designed such that the distance between them is less than one thousand base pairs. Once the primers are demonstrated to be allele-specific, they can be used for both allele-specific transcript abundance measurement when paired with a first primer homologous to a region in the transcript to PCR amplify cDNA, as well as allele-specific sequencing when paired with a second primer to PCR-amplify genomic DNA. In some preferred embodiments, the second primer is on the other side of a polymorphism of interest.

Those of skill in the art will recognize that the methods provided herein can be applied to the identification of polymorphisms for other biological states.

In preferred embodiments, DNA regions analyzed to provide polymorphisms include regions affecting transcription regulation, protein function, post-transcriptional processing, and/or protein-protein binding, including those in the 5′ regulatory region, those in the 3′ UTR, translated region, and 5′ UTR of the coding region. For example, sequences for DNA binding and heterodimer formation can be analyzed for polymorphisms indicative of BC. Additional details are provided in the Examples below.

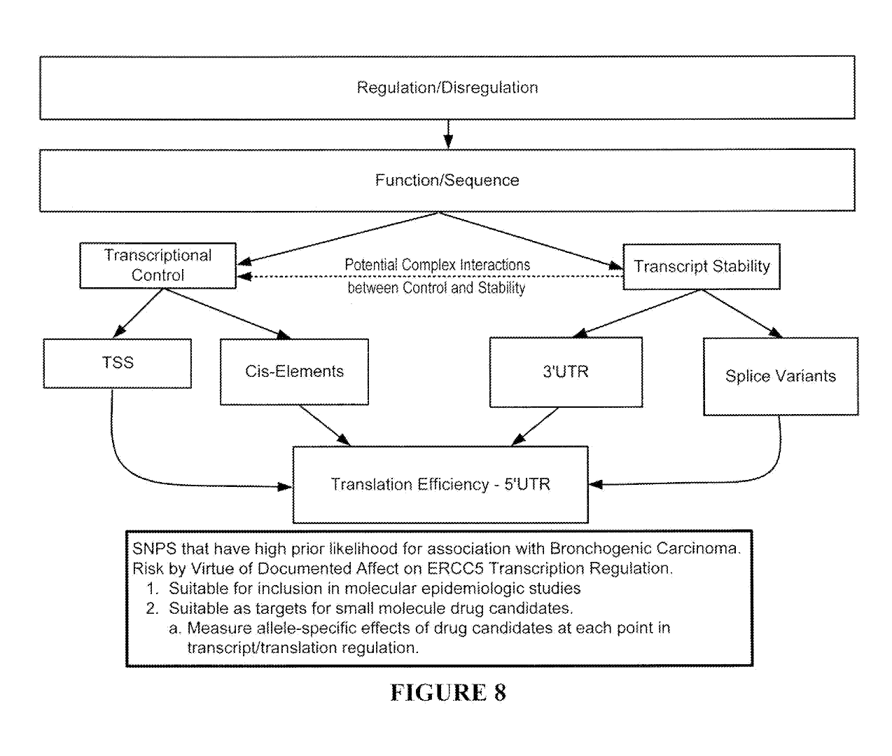

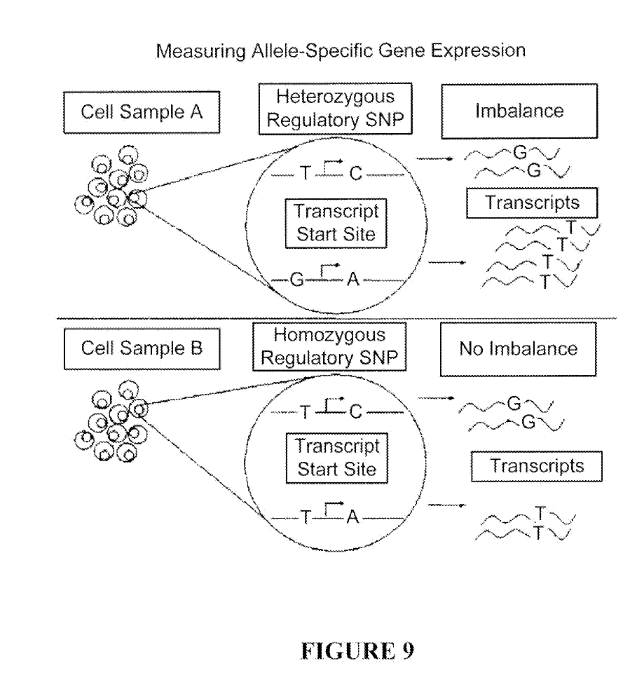

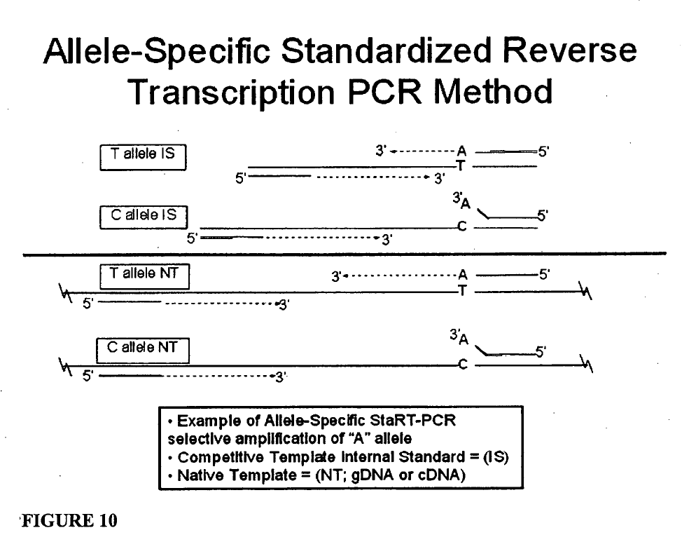

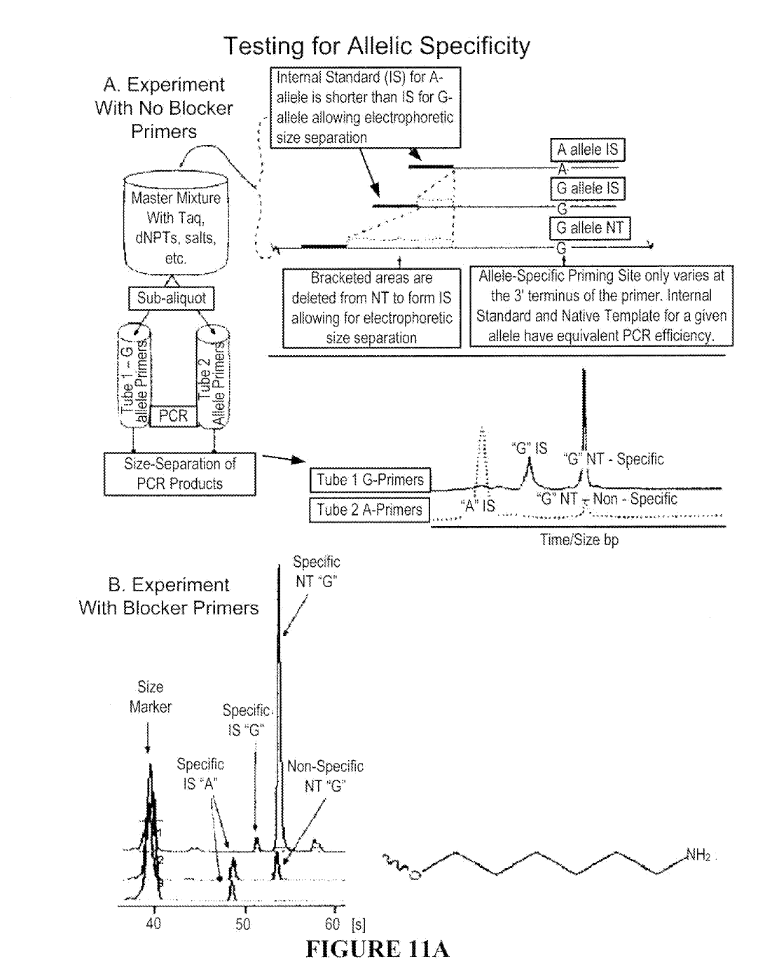

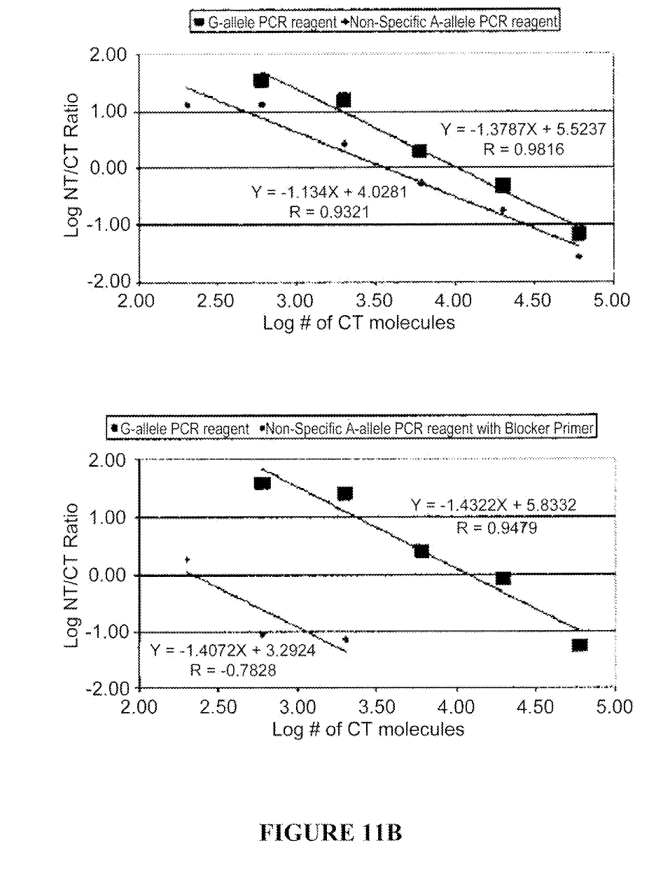

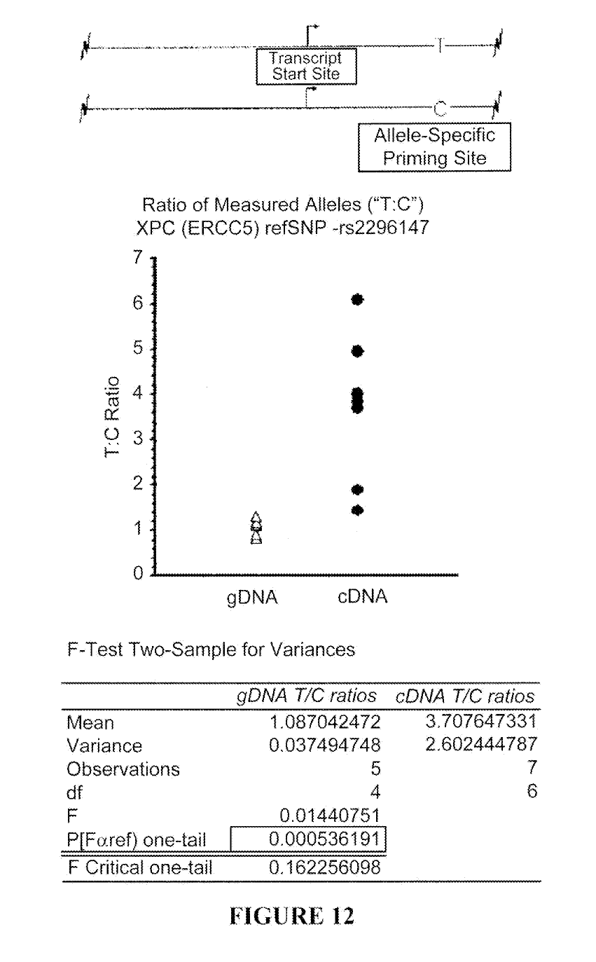

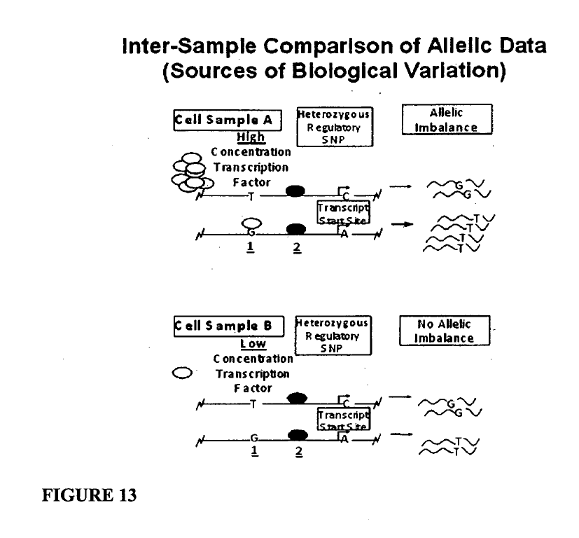

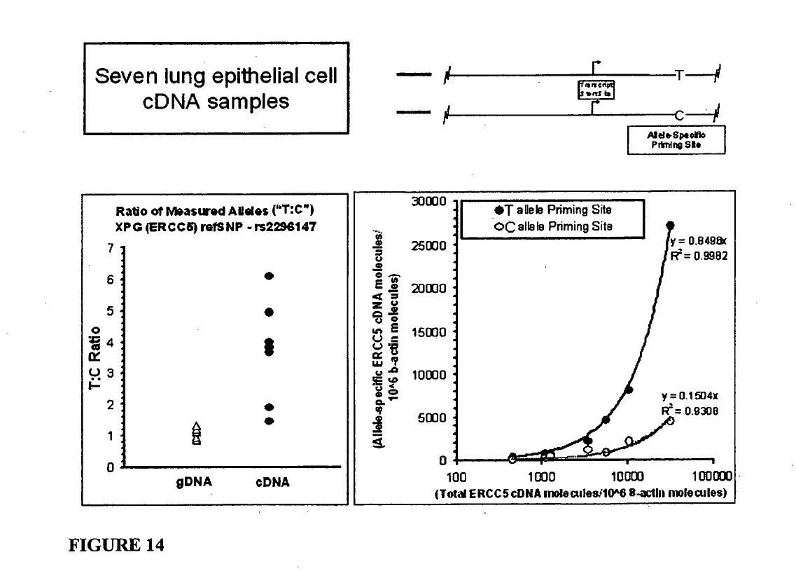

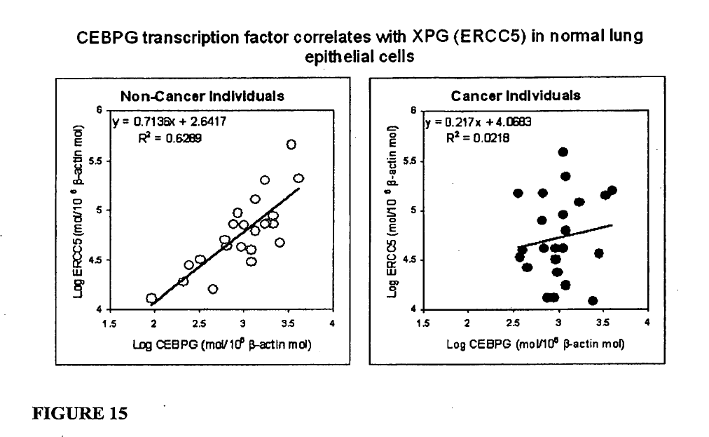

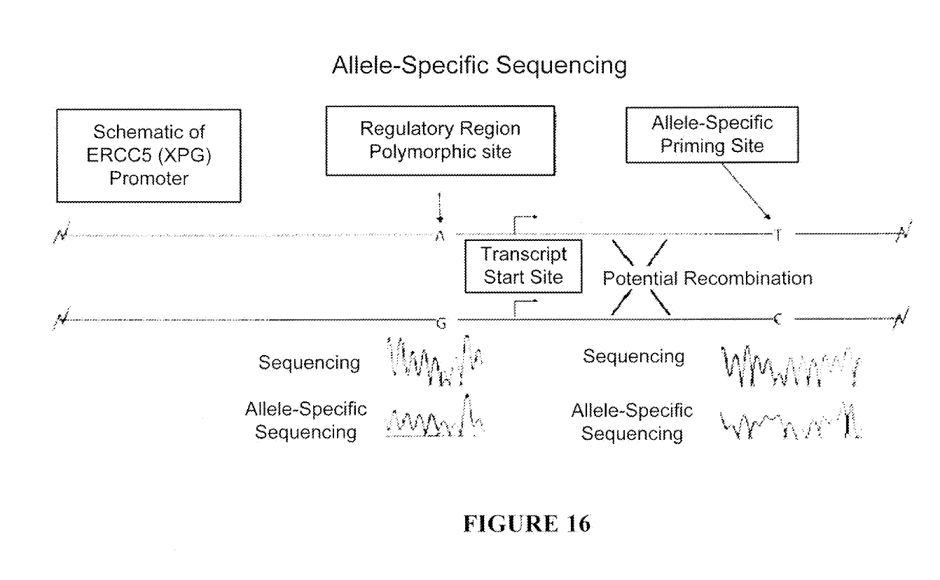

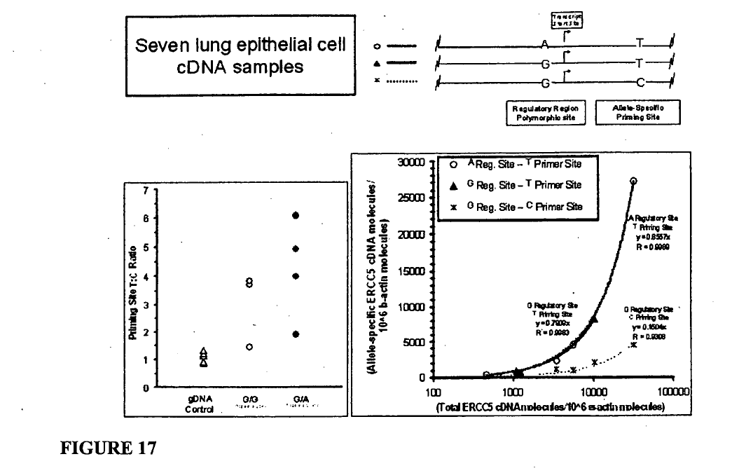

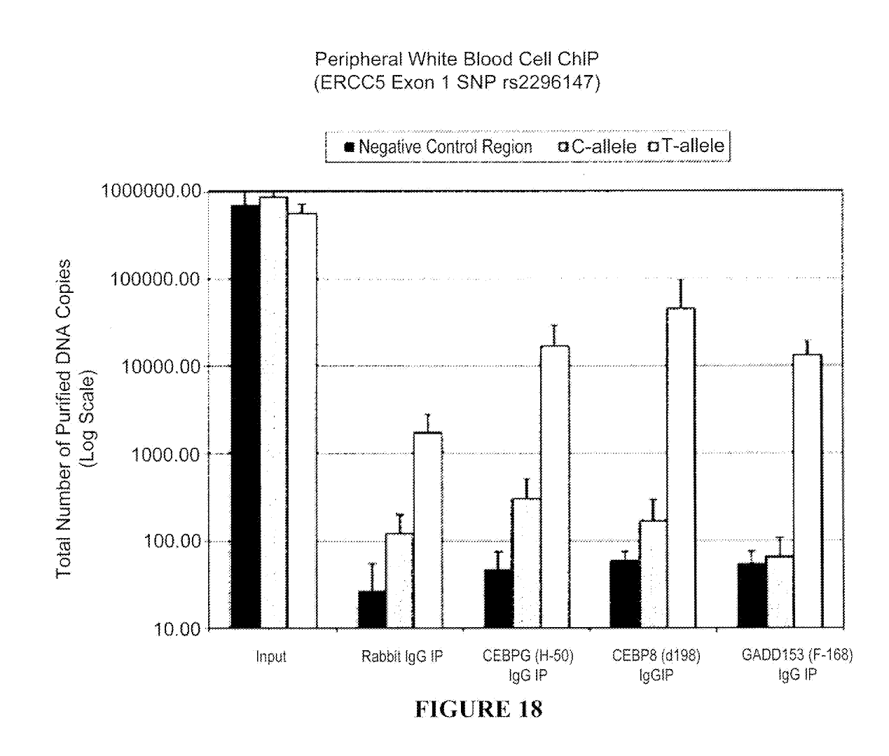

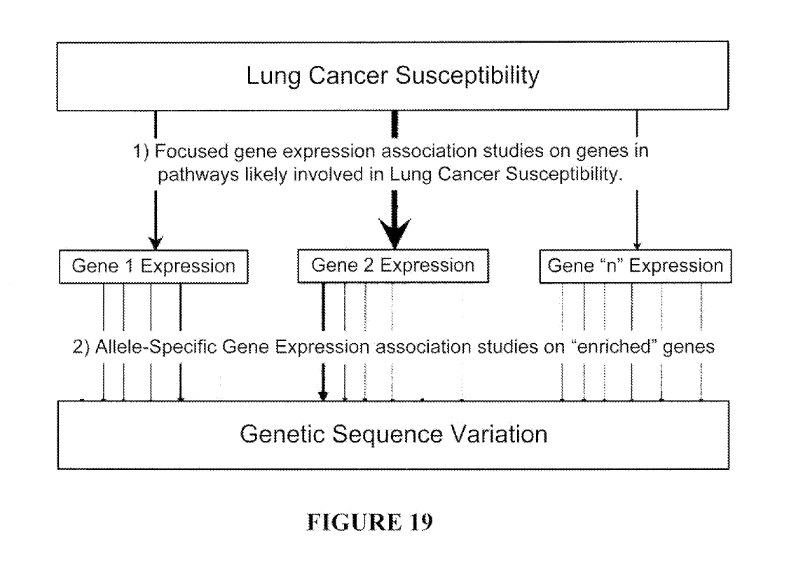

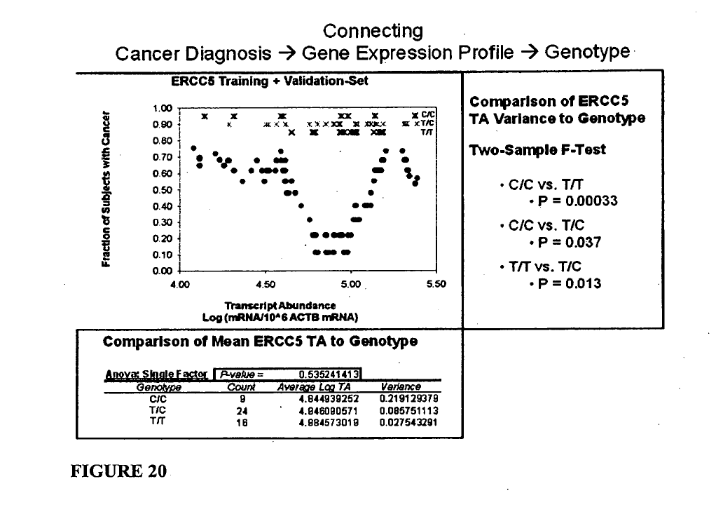

As illustrated in FIG. 8, according to the paradigm used in this study, LASTAS has multiple applications. By assessing allele-specific transcript abundance among many individuals, each with a different allelotype in the regulatory region, it is possible to determine the relative importance of each polymorphic site in regulating the gene. Many different possible mechanisms of gene regulation may be evaluated on an allele-specific basis. This will enable identification of polymorphic sites that alter regulation in vivo. Polymorphisms identified to alter regulation will have high prior likelihood for involvement in risk determination and determining beneficial or adverse response to pharmaceuticals. In addition, such polymorphisms documented to have effect on regulation are excellent targets in development of small molecule drug candidates.

In some embodiments, the invention provides kits for assessing the effect of DNA polymorphisms on regulation of transcription. In one embodiment, such a kit comprises allele-specific primers, primers for amplifying native templates, and third primers in the 5′ non-transcribed, intronic, or 3′ non-transcribed region of a genomic DNA template of said native template.

In some embodiments, kits and methods described herein provide more accurate identification of those at risk for BC and/or COPD, compared to traditional methods. More accurate identification of those at risk for BC and inclusion of such individuals in chemoprevention and/or early detection studies can lead to improved efficacy. Those of skill in the art will recognize that the methods provided herein can be applied to the diagnoses of other cancer-related conditions and/or other lung-related conditions, e.g., other cancer-related conditions and lung-related conditions provided herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and compositions within the scope of these claims and their equivalents be covered thereby.

All publications, patents, and patent applications mentioned in this specification are herein incorporated by reference to the same extent as if each individual publication, patent or patent application was specifically and individually indicated as being incorporated by reference.

### Examples

### Example 1

### Collection of NBCI and BCI Samples

Normal bronchial epithelial cell (NBEC) and peripheral blood samples can be obtained from patients and a portion of each sample can be used to in the Examples described herein. Individuals can be recruited from among patients who are undergoing diagnostic bronchoscopy. Some indications for bronchoscopy include coughing up of blood, chronic cough, pneumonia resistant to antibiotics, and need to remove a foreign body. Some of these patients may be diagnosed with bronchogenic carcinoma (BCI), while others may have non-neoplastic conditions (NBCI). The age of these patients may range from approximately 20 to approximately 90, with most participants being between the ages of about 60 and about 75. NBEC samples are obtained according to previously described methods. See, e.g, Benhamou S. et al., Carcinogenesis, 8: 1343-1350 (2002).

From each patient, NBEC samples and 20 ml of peripheral blood can be collected and processed, e.g., as previously described (Willey et al, 1997; Crawford et al, 2000). Approximately 10-15 brush biopsies can be obtained from normal appearing mucosa at approximately the tertiary bronchi. If the patient has a local pathological condition, such as pneumonia, trauma, or BC, the brushes can be taken from the opposite side.

After each bronchoscopic brush biopsy, the brush can be swirled in approximately 3 ml of ice cold saline to dislodge and retrieve the cells. Approximately 500,000 to 1 million cells can be obtained with each brush. Thus, 10 brushes can yield about 5-10 million cells. These cells in 3 ml of ice cold saline can be divided up for extraction of RNA and protein and preparation of slides for IHC and FISH. Approximately 5 million cells can be used for nuclear extract protein extraction (see, e.g., Dignam et al, Nucleic Acid Research 11: 1475-1489 (1983)). This can yield approximately 100 ug of nuclear extract for EMSA and Western hybridization analyses. Approximately 1 million cells can be used for RNA extraction for the expression level measurements.

The 20 ml of peripheral blood may contain approximately 1-2×10⁸ white blood cells. Most of these cells can be used to produce nuclear extracts for surface plasmon resonance (SPR) experiments described below. About 1-2 million cells can be used for RNA extraction for expression level measurements, and another about 1-2 million can be used for DNA extraction for sequencing studies.

Buccal and nasal epithelial samples can also be collected from BCI and NBCI providing bronchoscopic brush samples and peripheral blood samples for SNPs. Buccal and nasal epithelial samples can be obtained by brushing of the inside of the mouth or the nose. The buccal epithelial cell samples from BCI and NBCI can be handled in a similar way as the NBEC samples, as provided above.

### Example 2

### Determination of the Reproducibility and Robustness of the Preamplification StaRT-PCR™ Method.

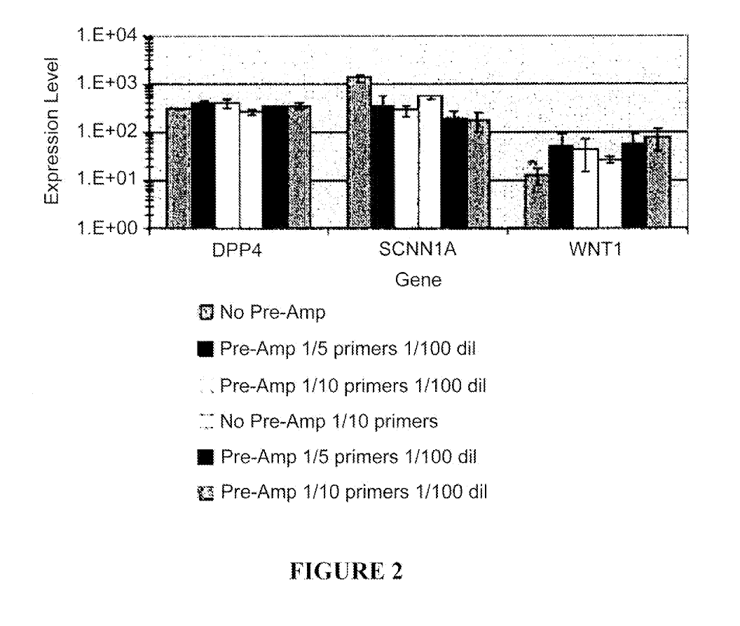

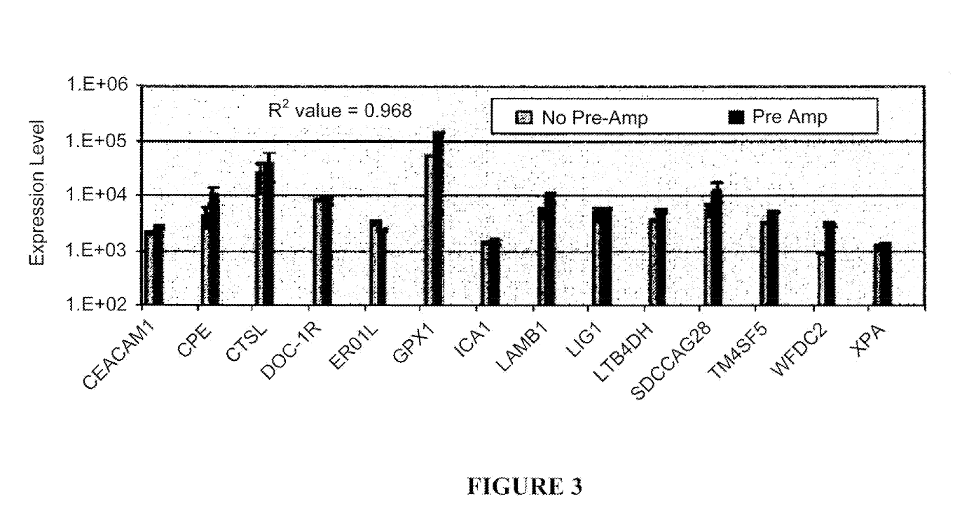

To determine the reproducibility and robustness of the previously published preamplification (Pre-Amp) StaRT-PCR™ method, levels of three genes expressed at low level (DP₄, SCNN₁A, and WNT₁) were measured in Stratagene Universal Human Reference RNA (SUHRRNA), under multiple conditions. In each experiment the conditions included the typical no pre-amplification method as a control, or Pre-Amp with a 96-gene primer mixture. Two different primer concentrations (⅕ or 1/10 usual concentration) were used in Pre-Amp. The Pre-Amp PCR products were now at a sufficient concentration to allow thousands of individual quantification reactions. In this example, the Pre-Amp PCR products were diluted 10- or 100-fold prior to the second round of PCR. The measured expression levels for experiments that included 1:100 dilution of Pre-Amp products are shown in FIG. 2. Both 10-fold and 100-fold dilutions of the Pre-Amp products produced similar variability in the measured transcript levels. These results demonstrate that, even with low expressed genes, which are subject to random sampling error, replicate variation is at an acceptable level with the Pre-Amp protocol, both within and across experiments.

### Example 3

### Determination of the Utility of a Two Step StaRT-PCR in Allowing the Use of a Small Sample for SNAP Assays.

In preparation for analysis of a series of transthoracic FNA samples, the SUHRRNA sample was assessed undiluted, 10-fold diluted, or 100-fold diluted by No Pre-Amp or Pre-Amp protocol. The best conditions of 10-fold dilution of primer mixture and 100-fold dilution of Pre-Amp products (1,000-fold overall dilution) were used to repeat analysis of SUHRRNA against fourteen genes. The results shown in FIG. 3 were that an average CV of 15.4% was determined in no Pre-Amp protocol and 14.8% with Pre-Amp protocol. Also, the mean values for five genes that had been previously measured gave mean values that were within 15% of values previously obtained during the optimization experiment. These conditions were then used to assess clinical samples obtained by transthoracic FNA biopsy. These results demonstrate that two step StaRT-PCR™ allows significant decrease of sample consumption while expanding the assay repertoire per sample.

### Example 4

### Determination of the Feasibility of Using the OpenArray System in SNAP Assays.

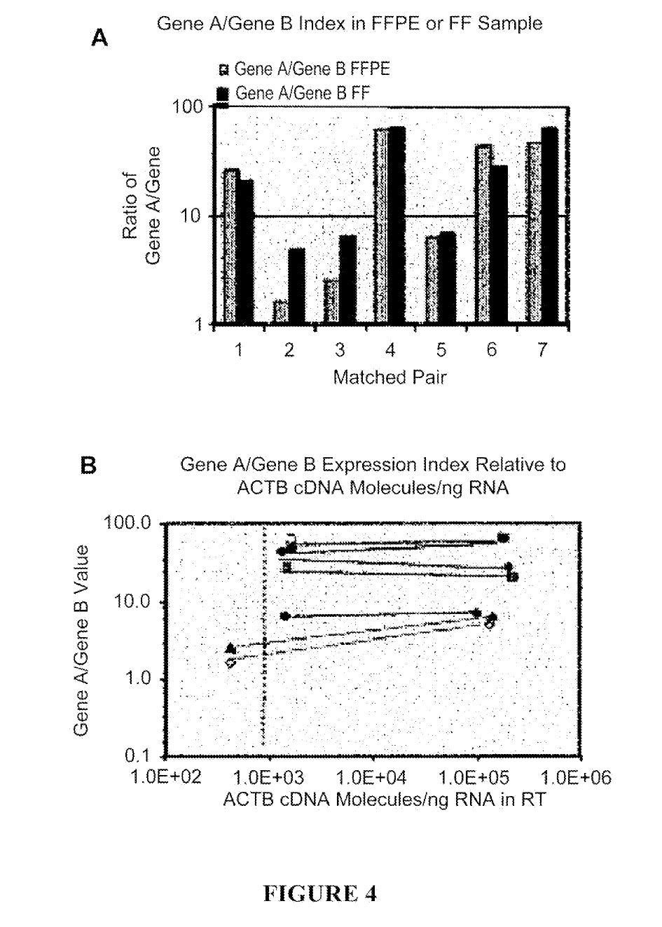

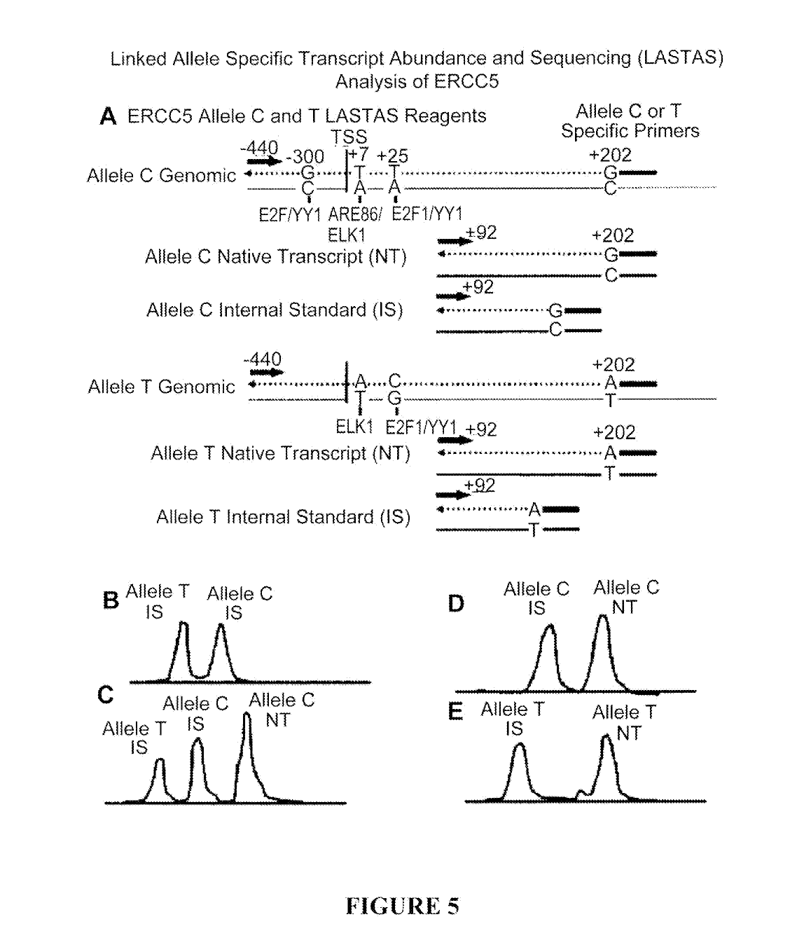

With careful reagent design, it is possible to obtain results from FFPE RNA samples that are less than 2-fold different from matched fresh frozen (FF) sample RNA samples. This was demonstrated in analysis of seven pairs of matched FFPE and FF RNA samples that were derived from seven cell line cultures that were split and either frozen or formalin fixed (FIG. 4, Panel A). In this case, the biomarker is a ratio of Gene A/Gene B. Ideally, the ratio of the FFPE Gene A/Gene B value to the FF Gene A/Gene B should be 1.0 for every matched pair. In FIG. 4A, the ratio varied 4-fold, from 0.4 in the second matched pair to 1.2 in the sixth matched pair. In contrast, the biological variation was greater than 13-fold among the FF samples. Thus, the biological variation exceeded the analytical variation between FF and FFPE RNA samples by more than 3-fold. The likely reason for the higher analytical variation in Gene A/Gene B ratio for matched pairs 2 and 3 is the higher level of RNA degradation in FFPE samples. Degradation was assessed from the number of beta-actin molecules obtained from 1 ng of RNA during reverse transcription, and the difference in Gene A/Gene B ration for the matched pairs was related to this measure of RNA degradation (FIG. 4, Panel B). Based on these results, a similar cut-off strategy comparing reference gene copies to input quantity of RNA to ensure RNA quality is employed.

### Example 5

Imaging and Analysis of Data from an OpenArray Assay.

Two color fluorescent images were collected following PCR using either BioTrove NT Imager, or third party microarray scanners (e.g., Tecan LS Reloaded). The FAM:VIC fluorescent ratio was plotted against internal standard Pre-Amp input quantity and inflection point from a sigmoidal curve fit (EC₅₀) was used to indicate native concentration. As a first pass examination of the OpenArray workflow, genomic human DNA was used to simulate the competitive PCR titration curve behavior. Similar to capillary electrophoresis and gel based methods used for StaRT-PCR™ endpoint measurements, TaqMan SNP assays are not analytically sensitive to a less than 10% native: control template, therefore, for this experiment the indicated log molar ratios <−1 and >1 used replicate homozygous genomic DNA instead of the indicated molar ratio. This experiment simulates the expected FAM:VIC ratio and variation for such samples. An analysis of eight technical replicates with sigmoid curve fits, resulted in an average of 0.055+/−0.007 EC_{50.} An 11% CV for technical replicates is reasable considering most of this variation can be accounted for by Poisson noise (˜6% given the 300 genomic template input) and will be greatly reduced when using the more highly expressed lung prognostic RNA targets.

### Example 6

### Preparation of C and T Allele-Specific Primers for the LASTAS Assay.

The approach schematically presented in FIG. 5 was used to successfully prepare C and T allele-specific primers (FIG. 6). In FIG. 6 are presented electropherograms of PCR products resulting from amplification of +202 heterozygous genomic DNA in the presence of equimolar mix of internal standards. The peaks other than those indicated by arrows are primer dimers or size markers. Thus, there was no non-specific amplification of C IS with T primer and no amplification of T IS with C primer.

### Example 7

### Use of Terminating Primers to Minimize Chances of Non-Specific Priming in LASTAS Assays.

Although allele-specificity is achievable under optimal conditions (FIG. 6), under extreme conditions of imbalance in alleles, the specificity may be tested. In order to minimize the chance that primers may bind and enable replication of the non-specific allele, allele-specific primers that have terminator groups that are not substrates for polymerase addition of NTP are used. The terminator primer for one allele (e.g. allele C) is included at normal primer concentration along with the extendible primer for the other allele (e.g. allele T). Any tendency for allele T primer to bind non-specifically to allele C in the reaction will be markedly diminished through competition from non-extendible terminator primer for allele C which will have perfect sequence homology. Although terminator primer for allele C may have small competitive effect on allele T primer binding, this will be controlled for by presence of allele T IS which will be affected the same way as the native template.

### Example 8

Sequencing Results from a LASTAS Assay.

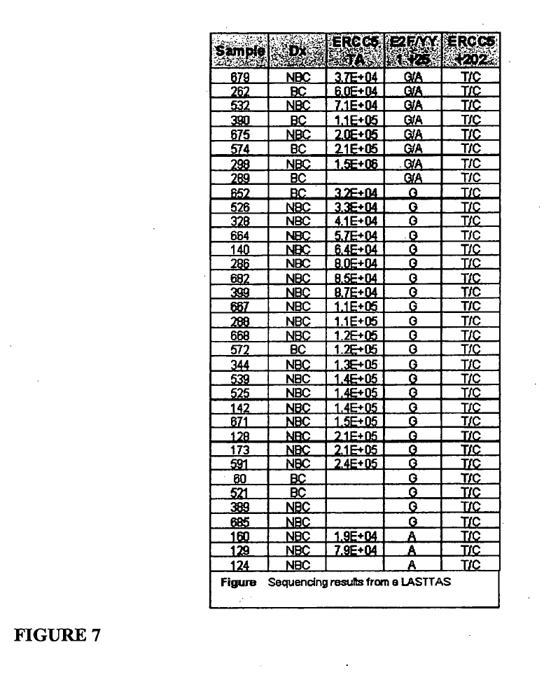

Sequencing of ERCC5 regulatory regions, including +202 and 500 bp upstream was conducted on about 85 of available samples. Some transcript abundance data from these samples has been published (Mullins et al., 2005). Of the 85 individuals sequenced, 35 were heterozygous at the +202 polymorphism (Table 2). Table 2 (FIG. 7) provides a list of these 35 BEC samples with sequencing result at both +202 and +25 polymorphisms, and annotation regarding whether they were from BC or non-BC subjects. Among these 35 heterozygous at +202, 8 were heterozygous at the E2F/YY1 (+25) site, 3 were homozygous A, and 24 were homozygous G. The ERCC5 expression level is also provided.

### Example 9

### Analysis of LASTAS Data.

Since it has to be assumed that each individual polymorphic site (such as E2F/YY1 site in Table 2) occurs in a different context (i.e., different allelotype at nearby or distant polymorphic sites that may affect regulation of the gene), determination of allele-specific affects at a particular polymorphic site requires analysis of groups of individuals. If the E2F/YY1 site plays an important role in ERCC5 regulation, the inter-individual variation in ratio of allele-specific transcript abundance at E2F/YY1 site among homozygote G individuals (i.e. rows 9 through 32 in Table 2) or homozygote A individuals (i.e. rows 33-35 in Table 2) will be small relative to the inter-individual variation in ratio of allele-specific transcript abundance among heterozygotes (i.e. rows 1-8 in Table 2). The degree of over-all affect contributed to regulation by a particular polymorphic site determines the relative difference in allele-specific ratio among heterozygotes relative to homozygotes. If the affect is small, a larger number of individuals will need to be in each group in order to detect significant difference. If the affect is large, a small group size will suffice. As experience is acquired, it may be possible to determine from the inter-individual variation among heterozygotes how much affect will be unaccounted for by the site being analyzed. This will guide in determining how much effort to put into assessing other polymorphic sires that may be involved in regulation. If variation among homozygotes is large and no polymorphic site within the sequenced gene is found to have variation above homozygotes, this will indicate presence of more distal polymorphic sites as responsible.

What is claimed:

1. A method for characterizing gene expression comprising:
(a) amplifying one or more native templates in a solution comprising a standardized mixture of internal standards and said one or more native templates to produce one or more first amplicon(s);
(b) amplifying said one or more first amplicons in a nanofluidic device, said nanofluidic device comprising at least one labeled probe that binds with greater affinity to either a first amplicon amplified from said native template or a first amplicon amplified from said internal standard;
(c) detecting signal from said at least one labeled probe bound to said native template and to said internal standard, and measuring a ratio of said native template to said internal standard based on said detected signals; and
(d) determining an amount of said one or more first amplicons from said ratio by multiplying said ratio by the number of copies of said standardized mixture of internal standards.

2. The method of claim 1 wherein the native template is DNA.

3. The method of claim 1 wherein the native template is mRNA.

4. The method of claim 1 wherein the native template is cDNA.

5. The method of claim 1 wherein the amplification to produce said one or more first amplicon(s) is a competitive amplification of said native template and said standardized mixture of internal standards.

6. The method of claim 1 wherein at least two labeled probes are used, wherein a first labeled probe binds with greater affinity to said first amplicon amplified from said native template and wherein a second labeled probe binds with greater affinity to said first amplicon amplified from internal standard.

7. The method of claim 6 wherein said at least two labeled probes are fluorescently labeled. A method of claim 6 wherein said at least two labeled probes comprise different labels.

8. A method of claim 1 wherein a signal generated from said probe bound to said first amplicon amplified from said native template or said first amplicon amplified from said internal standard is detected and quantified.

9. A method to assess an effect of DNA polymorphisms on regulation of transcription comprising:
(a) amplifying a native template from an individual using a first allele specific primer that anneals to a first allele at a first polymorphic DNA region and a non-allele specific primer to produce a first allele-specific amplification product;
(b) amplifying a native template from said individual using a second allele specific primer that anneals to a second allele at a first polymorphic DNA region and said non-allele specific primer to produce a second allele-specific amplification product;
(c) determining a ratio of each allele-specific product to the other;
(d) amplifying a DNA template of said individual using said first allele-specific primer for the first polymorphic DNA region and a second non-allele specific primer to produce a third amplicon spanning a sequence that contains a first allele at a second polymorphic region that is collinear with the first allele at the first polymorphic region;
(e) amplifying a DNA template of said individual using said second allele-specific primer that binds to the second allele at the first polymorphic site and said second non-allele specific primer to produce a fourth amplicon spanning a sequence that contains a second allele at said second polymorphic region that is collinear with said second allele at said first polymorphic region;
(f) determining a sequence of said third or fourth amplicon; and
(g) using said sequence to assess an effect of said second DNA polymorphic region on regulation of allele-specific transcription measured through allele-specific cDNA priming of said first polymorphic region.

10. The method of claim 9 wherein said first and second allele specific amplicons span a polymorphic locus putatively responsible for regulating transcription, translation, splicing, and/or degradation.

11. The method of claim 10 wherein said sequence collinear with said first polymorphic region is in the 5′ non-transcribed, intronic, or 3′ non-transcribed region of a DNA template.

12. The method of claim 10 wherein said DNA template is amplified with said first allele-specific primer for said first polymorphic site and a second primer that spans a second polymorphic locus in the 5′ non-transcribed region, 3′ non-transcribed region, or an intron.

13. The method of claim 9 wherein one or more internal standards corresponding to each allele is generated to ensure specificity of the allele-specific primers comprising:
a. synthesizing a primer for production of a first shortened allele-specific internal standard corresponding to the native template in step (a) of claim 9;
b. synthesizing a primer for production of a second shortened allele-specific internal standard corresponding to the native template in step (b) of claim 9;
c. producing a serial dilution of said first internal standard and combining it with a constant amount of the native template in step (a) of claim 9 to ensure allele-specificity of the primer; and
d. producing a serial dilution of said second internal standard and combining it with a constant amount of the native template in step (b) of claim 9 to ensure allele-specificity of the primer.

14. The method of claim 13 wherein said first and second internal standards are mixed together at known concentrations relative to each other.

15. The method of claim 14 wherein said first and second internal standards are mixed with a known concentration of an internal standard for one or more genes.

16. The method of claim 15 wherein said internal standard for one or more genes is a loading control.

17. The method of claim 9 further comprising measuring the transcript abundance of each allele relative to a known quantity of a corresponding internal standard.

18. The method of claim 9 wherein the native template is DNA.

19. The method of claim 9 wherein the native template is mRNA.

20. The method of claim 9 wherein the native template is cDNA.

21. A kit for assessing the effect of DNA polymorphisms on regulation of transcription comprising:
a) a first allele-specific primer that anneals to a first allele at a first polymorphic region in one or more native templates and a second primer suitable for PCR amplification;
b) a third allele-specific primer that anneals to a second allele at said first polymorphic region;
c) a fourth primer that binds to a nucleic acid sequence and in combination with said first allele-specific primer or said third allele-specific primer produce an amplicon that spans a DNA sequence containing a second polymorphic region that is a collinear transcribed sequence, 5′ non-transcribed sequence, an intronic sequence, or a 3′ non-transcribed sequence of a cDNA sample or genomic DNA sample; and
d) instructions for use.

22. The kit of claim 21 wherein said instructions disclose the use of allele-specific primers for amplifying each native template in a sample from an individual, and the third primer in the amplification of a genomic DNA native template of said sample.
